(19) United States
(12) Patent Application Publication
(10) Pub. No.: US 2013/0014671 A1
(43) Pub. Date: Jan. 17, 2013

(54) ANTIMICROBIAL DENTAL MATERIAL

(71) Applicant: Yu-Hong Tzeng, Taipei City (TW)

(72) Inventor: Yu-Hong Tzeng, Taipei City (TW)

(21) Appl. No.: 13/180,465

(22) Filed: Jul. 11, 2011

(51) Int. Cl. A61K 6/04; A61Q 11/00

(52) U.S. Cl. 106/15.05

(57) ABSTRACT

An antimicrobial dental material is characterized in: adding about 0.1˜0.5 weight % of antibacterial metal particles to a zirconia (ZrO₂) powder substrate of antimicrobial dental material; wherein after completely mixing and sintering thereafter the antibacterial metal particles are naturally diffused in the zirconia substrate, so that antimicrobial dental material for manufacturing implants of dentures or prostheses or any other oral reconstruction is available.

### FIELD OF THE INVENTION

The present invention relates to an antimicrobial dental material, particularly to an antimicrobial dental material which applies to manufacturing dentures or prostheses or any other oral reconstruction.

### BACKGROUND OF THE INVENTION

In previous dental reconstrution technology, metal has been used as material of dentures or prostheses. It has the disadvantage that metal dentures or crowns possess another color as that of the nature teeth, which causes a visible contrast to teeth color.

For aesthetic reasons dentures or prostheses made of glas or alumina ceramics have been introduced. Thereby, dentures and crowns of similar color as natural teeth are produced. However, glass and alumina material are fragile, having unreliable strength, so that safety is compromised.

Most of available dentures or prostheses of ceramics are made of zirconia material. Zirconia (ZrO₂) has not only high strengh, high hardness, temperature resistance and corrosion resistance as well as high chemical stability, it also possesses a higher tenacity then usual ceramics and very high biokompatibility as well. Therefore zirconia is widely applied to medicine. The chemical properties of zirconia are very stable. After calcining, it is especially inactive. For inceasing the stability of zirconia, yttria are added. Since zirconia has a similar color like natural teeth, it fulfills asthetic requirement of dentures and prostheses.

Although zirconia has the above-mentioned advantages, it does not prevent bacterial adhesion which resultes in denture plaque and periodontal disease. The main object of the present invention is to provide an antimicrobial dental material, which improves antibacterial properties of zirconia to obtain an optimal dental material.

Disinfection by metal ions is one of the feasible dental antimicrobial methods, since metal ions in very low concentrations destroy membrane activity or cell protoplasm enzymes and kill bateria.

Effects of metal ions on various harmful bacteria are different. Known antibacterial mental ions include precious metals like silver, gold, platinum, palladium and iridium; as well as base metals like titanium, copper, tin, antimony, bismuth and zinc.

The antibacterial effect of released ions from particulate antibacterial materials has been proven and widely applied to technology of medical equipment. Yet usage of antimicrobial metal materials for dentures or prostheses is unknown.

Besides, in conventional technology of dental material, surface treatment is mostly used, wherein a layer of antimicrobial material is set on the surface of dental material. For example, U.S. Pat. No. 6,267,590 (ANTIMICROBIAL DENTAL PRODUCTS) teaches setting of a layer of antibacterial means on the surface of dental material.

Furthermore, in the technology of antimicrobial ceramics, antibacterial properties are obtained through antibacterial material set on surface of ceramics. For example Taiwan Patent No. 528741 provides a method of mixing silver particles in ceramics glaze. so that the ceramics surface possesses antimicrobial glaze. Taiwan Patent no. I 248926 discloses a method of surface bathing, so that a solution with silver particles penetrates a ceramics surface. Therefore, in the known technology as mentioned above, only antibacterial properties on the surface are obtained, but not in the substrate of ceramics.

All the conventional technology as mentioned is through surface treatment to get improved antibacterial poperties. However, applying of dental material for dentures or crown implanting results in the following problems:

1. An antibacterial layer or sintered glaze of silver or copper metal ions, which is suspended on the surface of dental material, possibly peels off due to interface effects. In particular, dentures or prostheses endure strong biting forces, so the layer or sintered glaze of antimicrobial material can very easily peel off.

2. A layer of silver or copper particles on dental material releases ions, which are harmful for the human body by excess absorption.

Antimicrobial ceramics, antibacterial metal particles in conventional technology are usually mixed with polymer dielectric material before being added to a substrate, for preventing “bridge phenomenon”. So even mixing in the substrate is not available. Thereafter polymer dielectric material will be volatilized in high-temperatural sintering and removed.

However the mixing method of conventional antibacterial particulate material results in volatility of polymer dielectric material. Toxic gases and remains in the substrate are harmful for the human body if this method is applied on dental material. Therefore, conventional antimicrobial technology is unsatisfactory when applied to manufacturing dental material.

### SUMMARY OF THE INVENTION

The main object of the present invention is to provide an antimicrobial dental material, which possesses excellent antibacterial properties, has no problem with peeling off of layers, and is usable as material for dentures or prostheses as well.

For achieving above objects, the present invention provides an antimicrobial dental material, which is used as antimicrobial material of dentures or prostheses or other oral reconstructions, through adding 0.1˜0.5 weight % of antibacterial metal particles or particles of metal compounds in zirconia powder, thereafter sintering the mixture, wherein the particles are difussed in a zirconia substrate.

The antibacterial particles as mentioned above are metal materials (like silver, copper, titanium) or metal compounds (like titanium nitride), as well as non-metallic meterials, like silicon, which will release ions for antibacterial effect.

By mixing zirconia and the antibacterial metal particles, yttria or other stabile materials can be added, for geting improved mechanic stability of zirconia.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 is a flow chart of manufacturing the antimicrobial dental material of the present invention.

Appendix 1 shows a sintering temperatural curve of the antimicrobial dental material of the present invention.

Appendix 2 shows a scanning electron microscopy (SEM) of the antimicrobial dental material of the present invention after sintering, wherein 0.3 weight % of silver, copper and titanium is added in the zirconia.

Appendix 3 shows a comparison of hardness tests of the antimicrobial dental material of the present invention after sintering, wherein 0.1˜0.5 weight % of silver, copper and titanium is added in the zirconia.

Appendix 4 shows a SEM of antibacterial test as well as a comparison of antibacterial properties of the antimicrobial dental material of the present invention after sintering, wherein 0.3 weight % of silver, copper, titanium are added in the zirconia.

### DESCRIPTION OF THE PREFERRED EMBODIMENTS

The antimicrobial dental material of present invention comprises antibacterial metal particles releasing ions, which can be used as dental materials for manufacturing denture, crown or implant.

The antimicrobial dental material' of present invention comprises zirconia (ZrO₂) powder as a substrate, to which about 0.1˜0.5 weight % of antibacterial metal particles are added. The antibacterial metal particles are altered from metals (like silver, copper, titanium). After mixing the antibacterial metal particles with the zirconia powder and subsequent sintering, material for manufacturing dentures or prostheses or any other oral dental reconstruction is obtained.

When antibacterial metal particles are added to zirconia powder, ions released form the particles bring about an antibacterial effct. Since antibacterial metal particles are coated within zirconia powder, a dose of realeased ions is under control. Releasing excess metal ions, which are harmful for human body, is prevented.

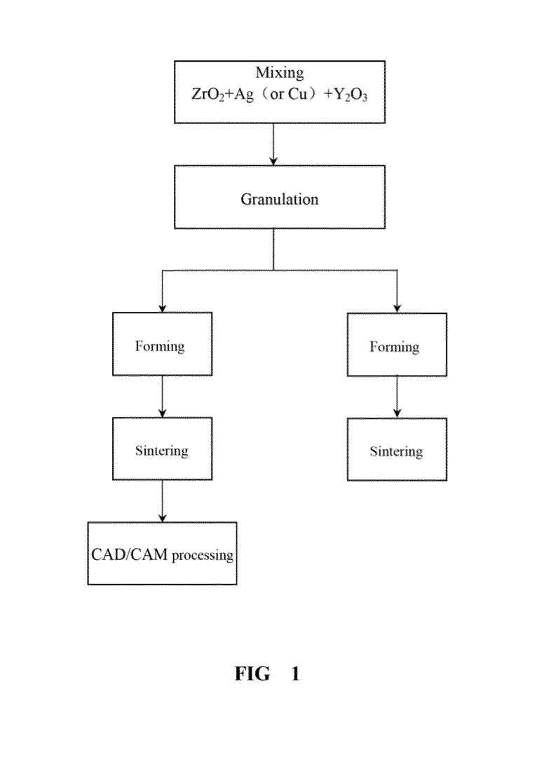

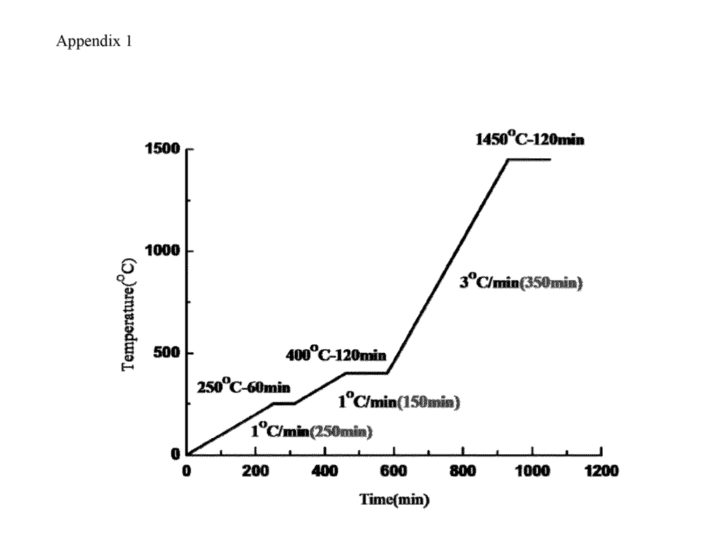

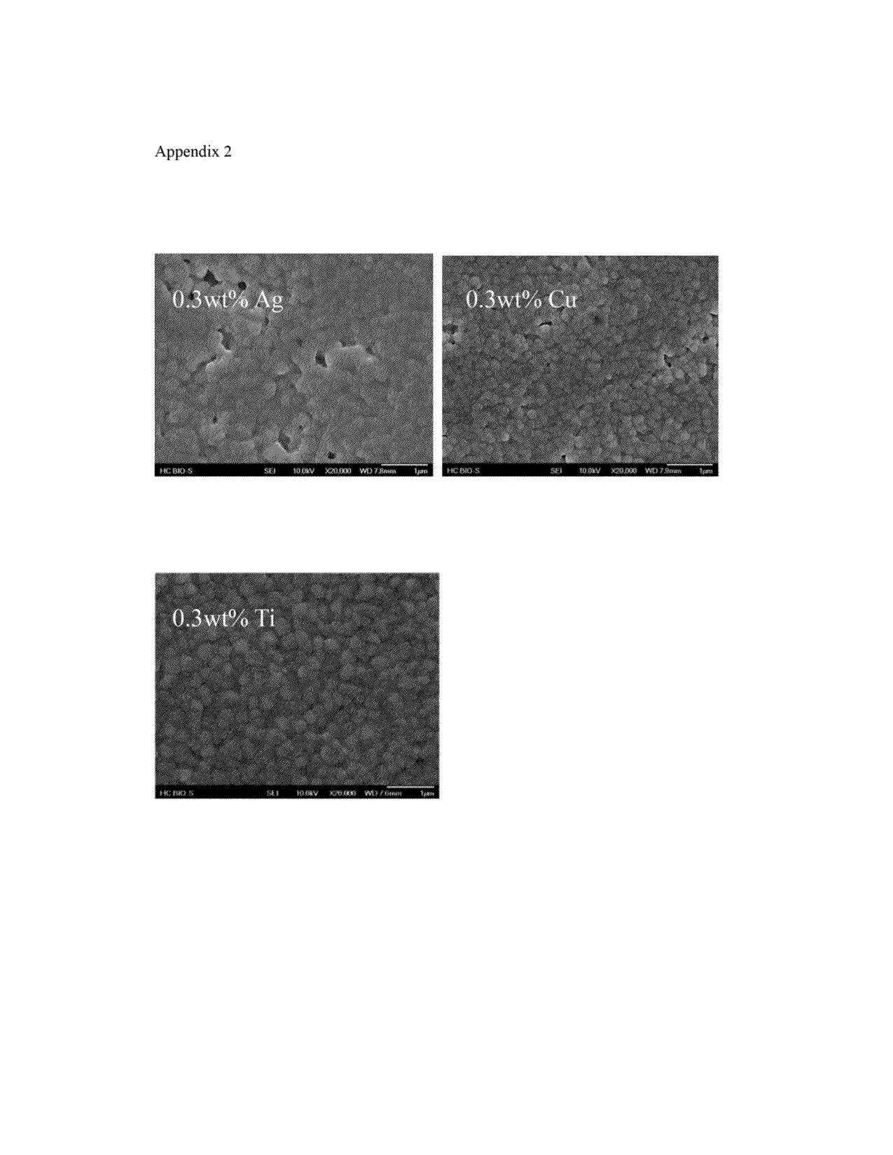

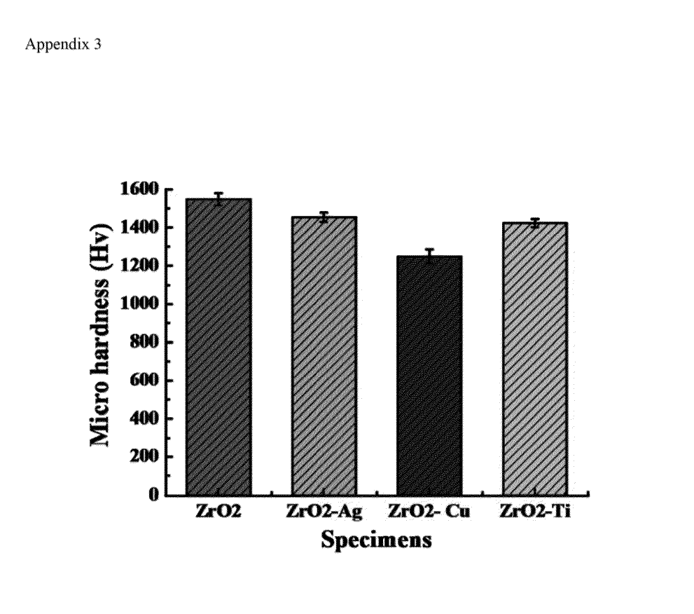

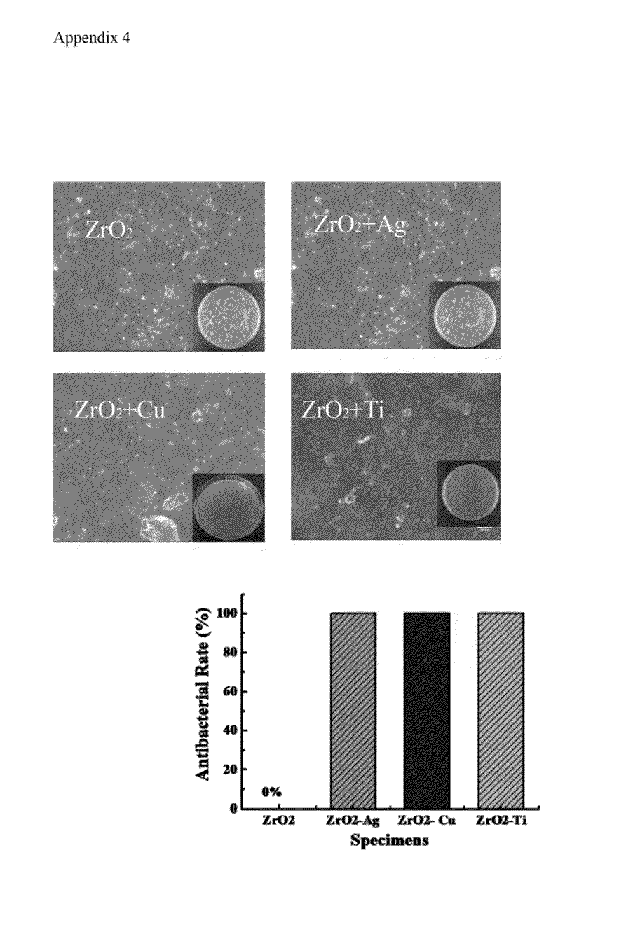

FIG. 1 shows a flow chart for manufacturing the antimicrobial dental material of the present invention: adding antibacterial metal particles to zirconia powder; completely mixing the particles and powder; and sintering.

During mixing and sintering, no dispersant for avoiding “bridge phenomenon” needs to be added, so that no dispersant with incomplete volatility remains in the zirconia substrate.

In commercially available metal particles, dispersant is usually added for preventing the bridge phenomenon. However, the dispersant, due to its tiny dose, is completely volatilized with no remains. Thus the antibacterial metal particles of present invention are commercially available. No special treatment ist necessary.

The method for a even distribution of the antibacterial metal particles of the present invitation, wherein sintering of zirconia with the particles in high temperature will result the metal particles naturely difused in the zirconia substrate. No dispersant is required.

After mixing zirconia with antibacterial metal particles, the mixture is granulated to preliminary masses. As the granulation proceeds, the zirconia powder is heated to 200-400° C., so that the antibacterial metal particles will be evenly distributed in the zirconia powder.

After processing the granulation, the granulated zircona is inserted into a sintering furnace. Appendix 1 shows the sintering temperatural curve during application of the method of the present invention. In the embodiment mentioned above, at a beginning phase, every minute the mixture is heated by one degree, until 250° C. are reached; a waiting period of 60 minutes ensues; every minute the mixture is heated by one degree, until 400° C. are reached; a waiting period of 60 minutes ensues; every minute the mixture is heated by three degrees, until 1450° C. are reached; a temperature of 1450° C. is kept for two hours; finally, heating is stopped, so that the sintered zirconia substrate naturally cools down.

The zircona powder is already be formed in the shape of dentures or prostheses, before sintering. Alternatively, the zircona powder is sintered into cubes, then cut using a CAD/CAM system into required shapes of dentures or prostheses.

Choosing of antibacterial metal particles of present invention depends on its antibacterial capacity, material costs and availability. Toxicity generating metal materials should be prevented. Metal materials of excellent antibacterial properties which release ions include: silver, gold, platin, palladium, iridium, titanium, copper, tin, antimony, bismuth and zinc. However regarding material costs, availibility and savety, it is preferable to use silver, copper and titanium in the present invention.

The substrate of zirconia powder can furthermore be mixed with stable materials like yttria for increasing stability afer sintering.

Experiments have shown, regarding factors of sufficient antibacterial effect, hardness and surface density keeping as well as non-excess ions releasing, that a preferable proportion of added antibacterial metal particles of the present invention is 0.1˜0.5 weight %. Thereof, 0.3 weight % is optimal.

Appendix 2 shows a scanning electron microscopy (SEM) map after adding of 0.3 weight % of silver, copper and titanium particles in the zirconia powder. Comparing the antimicrobial dental materials of the present invention with the zirconia ceramics without adding antibacterial metal particles after sintering shows that densities thereof are hardly different.

Appendix 3 shows a comparison of hardness values of the antimicrobial dental materials of the present invention after sintering, wherein 0.1˜0.5 weight % of silver, copper and titanium are added to the zirconia. There the zirconia with antibacterial metal particles possesses a hardness of over 1200 HV (Vickers Hardness) after sintering, which is hardly different from original zirconia ceramics.

Appendix 4 shows analyses of antibacterial properties of the antibacterial metal particles of the present invention, wherein 0.3 weight % of silver, copper and titanium have been added to the zirconia substrate. In the used test method. zirconia ceramics lies in a petri dish of 37° C. for 24 hours, the bactericidal property can reach over 99.8%. It is obvious that the present invention has excellent antibacterial effect.

The test as mentioned above has demonstrated that the antimicrobial dental material of the present invention, while having similar mechanical property as konventional zirconia ceramics, has an excellent antibacterial effect. Therefore it is a optimal dental meterial for manufacturing dentures or prostheses.

The antimicrobial dental material of the present invention has not only an excellent antibacterial effect, due to antibacterial silver or copper ions added to zirconia powder. Since the antibacterial metal particles are coated in zirconia powder, it can also prevent ions releasing from the particles as well as peeling off due to intersurface effects. Furthermore, neither a surface treatment nor a layer setting is performed.

1. An antimicrobial dental material, wherein antibacterial metal particles are added to a zirconia (ZrO₂) powder substrate, and subsequently said zirconia powder substrate and antibacterial metal particles are mixed completely and sintered.

2. The antimicrobial dental material of claim 1, wherein said antibacterial metal particles are altered from one of the metals silver, copper or titanium, or mixed from at least two of said metals.

3. The antimicrobial dental material of claim 1, wherein a proportion of 0.1˜0.5 weight % of said added antibacterial metal particles is added to said zirconia powder substrate.

4. The antimicrobial dental material of claim 2, wherein a proportion of 0.1˜0.5 weight % of said added antibacterial metal particles is added to said zirconia powder substrate.

5. The antimicrobial dental material of claim 1, wherein a proportion of 0.3 weight % of said added antibacterial metal particles is added to said zirconia powder substrate.

6. The antimicrobial dental material of claim 2, wherein a proportion of 0.3 weight % of said added antibacterial metal particles is added to said zirconia powder substrate.

7. The antimicrobial dental material of claim 1, wherein yttria (Y₂O₃) is further added.

8. The antimicrobial dental material of claim 2, wherein yttria (Y₂O₃) is further added.

9. The antimicrobial dental material of claim 3, wherein yttria (Y₂O₃) is further added.

10. The antimicrobial dental material of claim 4, wherein yttria (Y₂O₃) is further added.

11. The antimicrobial dental material of claim 5, wherein yttria (Y₂O₃) is further added.

12. The antimicrobial dental material of claim 6, wherein yttria (Y₂O₃) is further added.
